(19) United States
(12) Patent Application Publication
(10) Pub. No.: US 2007/0218023 A1
(43) Pub. Date: Sep. 20, 2007

(54) Uses of melatonin in skin

(76) Inventors: Andrzej Slominski, Germantown, TN (US); Trevor W. Sweatman, Memphis, TN (US); Tobias W. Fischer, Lubeck (DE); Igor Semak, Minsk (BY)

Correspondence Address: Dr. Benjamin Adler;ADLER & ASSOCIATES, 8011 Candle Lane, Houston, TX, 77071, US

(21) Appl. No.: 11/708,832

(22) Filed: Feb. 21, 2007

Related U.S. Application Data
(60) Provisional application No. 60/775,177, filed on Feb. 21, 2006.

(51) Int. Cl. A61K 8/49

(52) U.S. Cl. 424/59

(57) ABSTRACT

Melatonin, which can be produced in the skin, exerts a protective effect against damage induced by ultraviolet radiation (UVR). The present study investigated the effect of UVB, the most damaging component of UVR, on melatonin metabolism in HaCaT keratinocytes and in a cell-free system. Four metabolites were identified by HPLC and LC-MS: 6-hydroxymelatonin, AFMK, 2-hydroxymelatonin (the main intermediate between melatonin and AFMK) and 4-hydroxymelatonin. Concentrations of these photoproducts were directly proportional to UVR-dose and to melatonin substrate content, and their accumulation was time dependent. The UVR-dependent increase of AFMK and 2-hydroxymelatonin was also detected in keratinocytes, where it was accompanied by simultaneous consumption of intracellular melatonin. Of note, melatonin and its two major metabolites, 2-hydroxymelatonin and AFMK, were also detected in untreated keratinocytes, neither irradiated nor preincubated with melatonin. Thus, intracellular melatonin metabolism is enhanced under exposure to UVR. The additional biological activity of these individual melatonin metabolites increases the spectrum of potential actions of the recently identified cutaneous melatoninergic system.

### CROSS-REFERENCE TO RELATED APPLICATION

This non-provisional application claims benefit of provisional U.S. Ser. No. 60/775,177, filed Feb. 21, 2006, now abandoned.

### BACKGROUND OF THE INVENTION

1. Field of the Invention

The present invention relates generally to the field of dermatology. More specifically the present invention relates to the metabolism of melatonin under the influence of UVR in skin cells and in cell-free conditions and supports a novel role of melatonin as protector of the skin against solar radiation.

2. Description of the Related Art

Melatonin is no longer considered exclusively a pineal hormone (1, 2), but a bioactive substance with extrapineal sites of synthesis (3-10). Thus, melatonin can act as a receptor independent autocrine and paracrine antioxidant (11, 12), as a direct radical scavenger (11, 13), as an immunomodulator (14), and as an antiaging (15, 16) and anticarcinogenic factor (17) in physiological and pharmacological concentrations (18).

An important peripheral target of melatonin is the skin where it is organized in a melatoninergic system, fully expressed in humans (9, 10, 19, 20) and rodents (21-23). Melatonin biosynthesis in the skin requires uptake of the essential amino-acid L-tryptophan and enzymatic formation of 5-hydroxytryptophan by tryptophan hydroxylase which is dependent on (6R) 5,6,7,8-tetrahydrobiopterin (6-BH4) (24, 25). Decarboxylation and further multistep-synthesis requiring arylalkylamine-N-acetyltransferase (AANAT) and hydroxy-indol-O-methyltransferase (HIOMT) produces melatonin (10, 19). Functionally, both cultured human keratinocytes and rodent melanoma cells have been shown to phenotype properties sensitive to melatonin (26-29) suggesting that melatonin, endogenous or exogenous could play a role in protection of the skin against environmental stressors such as ultraviolet radiation (UVR) (9). In fact, UVR is the most prominent causative factor in photo-aging and skin cancer and is likely responsible for the increasing incidence of UV related basal cell and squamous cell carcinoma (30, 31) as well as malignant melanoma (32) over the last decades. Being a strong protectant against UV-induced damage both in vitro (9, 10, 33, 34) and in vivo (35, 36), melatonin could ensure the survival of keratinocytes and their clonogenic capacity against UV-induced damage (37). Moreover, it has been shown in vivo that exogenous melatonin applied topically in a cream preparation can penetrate into the skin and build a depot in the upper layers of the epidermis to supplement the protective effects of endogenous melatonin (38).

Melatonin is a strong radical scavenger and able to protect the skin against oxidative damage and cell death induced by ultraviolet radiation (UVR). However, there is controversy on the direct effect of UVR on melatonin; while some have postulated that UVR causes production of melatonin phototoxic products (39), others have identified protective effects of UV induced melatonin degradants, classified as antioxidative or antiinflammatory (40-46). Of note, these studies were performed in cell-free systems or in cells not normally exposed to UVR such as neutrophils (42, 47) and macrophages (40). Obviously, the cell population of maximal interest concerning UVR exposure and its effects on melatonin are the skin keratinocytes, however, these have not been previously investigated. This is particularly important, because a melatoninergic system is fully expressed in the skin (9, 10). Hence, the present art is deficient in knowledge of the influence of UVB, the most damaging wavelength of UVR, on the molecule melatonin itself. The present invention fulfills this long-standing need and desire in the art.

### SUMMARY OF THE INVENTION

This invention provides a novel role of melatonin as a protector for the skin against solar radiation. Further the present invention defines a novel melatoninergic antioxidative system (MAS) of the skin where the UV-induced production of melatonin metabolites, act as strong antioxidants. In addition, the present invention demonstrates that the combination of endogenous melatonin with externally applied melatonin may successfully counteract the multiple processes of skin damage induced by UVR. Furthermore, this invention demonstrates the antiapoptotic mechanism of melatonin following UVR exposure in keratinocytes.

Thus, the present invention is directed to a method of protecting the skin, from the damaging effects of ultra-violet radiation consisting of applying topically an effective dose of a formulation comprising melatonin as the single active ingredient; where the application protects the skin from the damaging effects of ultra-violet radiation.

The present invention is further directed to a method of reducing photodamage to the skin of a subject consisting of administering a composition comprising melatonin to the subject in an amount effective to inhibit UVB-induced apoptosis of skin cells exposed to ultra-violet radiation.

The present invention is still further directed to a method of retarding development of skin cancer in an individual at risk consisting of

applying topically an effective dose of a formulation comprising melatonin; where the application retards development of skin cancer in said individual at risk.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows emittance spectrum of the ultraviolet transluminator (Biorad Model 2000) operating in normal and preparative mode. The maximum emittance is in the UVB range (280-320 nm).

FIG. 2 shows detection of melatonin metabolites in a cell-free system measured by HPLC: 6-hydroxymelatonin (product 1), 2-hydroxymelatonin (product 2), 4-hydroxymelatonin (product 3) and AFMK (product 4). Product levels increased after irradiation of melatonin solution (10⁻³ M) in direct proportion to UV-doses (25, 50, 100 mJ/cm²).

FIG. 3 shows baseline level (1) of AFMK (product 4) analysed by LC-MS in extracts of melatonin solution (10⁻³ M) which has been irradiated with increasing UVR doses of 25 (2) and 75 mJ/cm² (3).

FIG. 4 shows detection of 2-hydroxymelatonin and 4-hydroxymelatonin by LC-MS (SIM mode for m/z=249) after irradiation of melatonin solution (cell-free system) with 75 mJ/cm². UV absorption spectra of the products (identical to the standards) confirm the structure of the compounds (see insets).

FIGS. 5A-5F show identification of products 2 and 4. UV absorption curves of products 2 (RT 34 min) (A) and product 4 (RT 43 min) (D) are identical to absorption curves of corresponding standards of 2-hydroxymelatonin (inset in A) and AFMK (inset in D) analysed by HPLC. LC-MS analysis in SIM mode (m/z=249 and m/z=265) further confirms that products 2 and 4 are 2-hydroxymelatonin (B; arrow) and AFMK (E; arrow), respectively. Mass spectra of the products and their chemical structure are presented in C and F, respectively.

FIG. 6 shows AFMK (product 4) detected as photoproduct after irradiation of melatonin solution at the dose of 25 mJ/cm², peaking at the same retention time as synthetic AFMK standard.

FIGS. 7A-7C show kinetics of extracellular accumulations of 2-hydroxymelatonin (FIG. 7A), 4-hydroxymelatonin (FIG. 7B) and AFMK (FIG. 7C). HaCaT keratinocytes were preincubated with 10⁻³ M melatonin and exposed to UVR. Analysis of collected supernatants show that all products increased as early as 40 min after irradiation with 2- and 4-hydroxymelatonin remaining at high levels until 370 min post UVR, whereas AFMK showed a decrease after 190 min, indicative of further metabolism to AMK.

FIG. 8 shows relative levels of 2-hydroxymelatonin in HaCaT keratinocytes without melatonin preincubation. Irradiation with UVR (50 mJ/cm²) leads to a 3-fold increase in intracellular 2-hydroxymelatonin concentrations compared to control. Columns represent means of absolute AUC-values of absorbance peaks from two experiments. Values above columns represent percentage mean±SEM of unirradiated control.

FIGS. 9A-9B show detection of extra- and intracellular AFMK (FIG. 9A) and melatonin (FIG. 9B). The levels of AFMK were higher in keratinocytes preincubated with melatonin than in control cells (no melatonin added) (FIG. 9A). AFMK was also higher in supernatants that had been preincubated with melatonin, than without melatonin preincubation (FIG. 9A, insets). In both conditions and compartments, levels of AFMK increased after UV exposure. Melatonin, in contrast, showed reduced intracellular levels after UV exposure (FIG. 9B), whereas extracellular melatonin levels in the condition of melatonin preincubation showed no reduction (FIG. 9B, inset). Columns represent means of absolute AUC-values of absorbance peaks from two experiments. Values above columns represent percentage mean±SEM of unirradiated control.

FIGS. 10A-10C show relative levels of endogenous melatonin (FIG. 10A), AFMK (FIG. 10B) and 2-hydroxymelatonin (FIG. 10C) in HaCaT keratinocytes. Melatonin was detected in cells from control (no melatonin added) keratinocytes and its level decreased at 24 hrs, indicative of metabolic consumption (FIG. 10A). In contrast, intracellular levels of AFMK (FIG. 10B) and 2-hydroxymelatonin (FIG. 10C) increased after 24 hrs incubation.

FIG. 11 shows the melatoninergic antioxidative system (MAS) of the skin. Parallel to scavenging UVB-induced reactive oxygen species (ROS), namely hydroxyl radicals, melatonin is also transformed to 2-hydroxymelatonin, 4-hydroxymelatonin and consecutively to AFMK. AFMK is a potent free radical scavenger and therefore by itself capable of protecting the skin against lipid peroxidation, protein oxidation and oxidative DNA damage. The endogenous melatoninergic system can be supported by exogenous application of melatonin, which penetrates easily through the stratum corneum into deeper layers of the skin.

FIGS. 12A-12B show the protective effect of melatonin on UVR induced changes in keratinocytes morphology. Control represented by non-irradiated keratinocytes is show in (FIG. 12A). After irradiation with 50 mJ/cm² no immediate change was observed (0 hrs, upper panel), whereas at 24 hrs (mid panel) and 48 hrs after UV exposure cultures showed empty spaces, which were absent in melatonin treated keratinocytes (FIG. 12B). One set of images from one representative experiment out of three is presented.

FIGS. 13A-13D show UV induced changes in mitochondrial potential (ΔΨ) and its prevention by presence of melatonin. Representative images show the mitochondrial membrane potential of control (FIG. 13A), non-irradiated HaCaT keratinocytes incubated with melatonin [10⁻⁴M] (FIG. 13B) and keratinocytes, irradiated with UVB (50 mJ/cm²) in presence (FIG. 13D) or absence (FIG. 13C) of melatonin [10⁻⁴M]. Nuclear cross-sections were acquired using confocal microscopy. Mitochondrial membrane potential was indicated by JC-1 red fluorescence (left panels). The relative change in mitochondrial membrane potential was demonstrated by the shift from red to green fluorescence (middle panels) and expressed as a red to green ratio resulting in blue fluorescence (right panel). Bar −20 μm

FIGS. 14A-14B show changes in mitochondrial potential (ΔΨ) induced by UV (FIG. 14A) or H₂O₂ (FIG. 14B) were prevented by presence of melatonin. HaCaT keratinocytes were preincubated with melatonin at the concentration of 10⁻³ to 10⁻⁶ M (FIG. 14A) or 10⁻⁴ M (FIG. 14B) for 30 minutes (FIG. 14A, FIG. 14B) or 120 minutes (FIG. 14A) followed by irradiation with UVB (50 mJ/cm²) (FIG. 14A) or by treatment with H₂O₂ (1 mM, 60 minutes) (FIG. 14B). Graphs show quantification of mitochondrial potential (ΔΨ) expressed as ratio of J monomer/J-aggregate fluorescence (red/green) in different treating groups with lower values representing stronger reduction of membrane potential. *P<0.05 ** P<0.005,*** P<0.0005 vs UV treated cells (no melatonin) (Panel A). ** P<0.005,*** P<0.0005 vs H₂O₂ treated cells (no melatonin) (Panel B).

FIG. 15A-15H shows activation of initiator and effector caspases and PARP. Decreased activation of caspase 9 in melatonin treated samples compared to non-melatonin treated samples, represented by lower expression of cleaved form of casp-9 of 35 and 17 kDa is in (FIG. 15A, FIG. 15E). The UV-induced activation was stronger at 24 hrs vs. 48 hrs after UV exposure. The peak of the cleaved form of effector caspases 3 (17 kDa) (FIG. 15B, FIG. 15F) and casp-7 (20 kDa) (FIG. 15C, FIG. 15G) occurred at later time points (48 hrs post UV) and their activation was similarly reduced by melatonin. Activated PARP (89 kDa) was detected as early as 24 hrs after UV irradiation, and melatonin reduced its activation (FIG. 15D, FIG. 15H). Equal loading of proteins was confirmed by protein staining (PS) with Commassie-Blue. Densitometric analysis of immunoblot bands of casp-9 (FIG. 15E), casp-3 (FIG. 15F), casp-7 (FIG. 15G) and PARP (FIG. 15H) represented mean values and standard deviation from two to three experiments as presented in column diagrams.

FIG. 16 shows the interaction of melatonin with essential cellular pathways of apoptosis in UV-irradiated keratinocytes. Melatonin prevents reduction of mitochondrial membrane potential and consecutive activation of initiator caspases in the mitochondrial (casp-9) pathway at an early time point of UV-induced apoptosis (24 hrs), whereas the death-receptor mediated extrinsic pathway through casp-8 is not influenced by melatonin. Down-stream events such as activation of effector caspases occur at latter time points (48 hrs) and are also reduced in melatonin treated cells. PARP activation is prevented at 24 hrs in cells pre-incubated with melatonin, most likely through direct protection of DNA by melatonin, independently from the caspase pathway.

### DETAILED DESCRIPTION OF THE INVENTION

The present invention describes melatonin metabolism in cell-free systems and cultured keratinocytes in vitro. Additionally, the present invention also investigates, the effect of UVR exposure on this metabolism. The present invention demonstrates intense local metabolism of melatonin with generation of its metabolites namely, 6-hydroxymelatonin, 2-hydroxymelatonin, 4-hydroxymelatonin and N¹-acetyl-N²-formyl-5-methoxykynuramine (AFMK). Furthermore, the present invention demonstrates production of melatonin in keratinocytes incubated in media free of melatonin. Thus, the present invention uncovers a novel functional significance for the cutaneous melatoninergic system. In addition, the present invention provides a detailed analysis of the time-dynamic execution of UV-induced apoptosis on cell morphological level and on intrinsic and extrinsic apoptotic pathways as well as on mitochondrial membrane potential reduction, and its prevention by melatonin.

In humans, 6-hydroxymelatonin is the chief metabolite of circulating melatonin which is either endogenously produced by the pineal gland or of exogenous source by oral intake. Circulating melatonin is 6-hydroxylated through first-pass hepatic metabolism, further conjugated to 6-sulfatoxymelatonin and excreted in urine (49, 50). The present invention did not find 6-hydroxymelatonin as a major product of UV-irradiation, although it was detected in non UV-exposed keratinocytes and its intracellular levels decreased after 24 hrs of incubation. Thus, it is most likely, that keratinocytes do metabolize melatonin to 6-hydroxymelatonin and may even have the capability to conjugate 6-hydroxymelatonin to 6-sulfatoxymelatonin, which is further released extracellularly. Indeed, Maharaj et al. (44) identified 6-hydroxymelatonin and N¹-acetyl-N²-formyl-5-methoxykynuramine (AFMK) after exposing melatonin in a cell-free system to UV-VIS (visible wavelength) in a proportion of 1:2. In contrast, the present invention reveals 6-hydroxymelatonin to be only a minor product after UVR, which may be explained by the use of different UV wavelengths. However, the present invention confirms AFMK as the major product of melatonin degradation induced by selected UVR wavelengths. To date, AFMK has been detected only in the rat retina (51). In vitro, AFMK is generated by oxidation of melatonin, for example by reactive oxygen species (ROS) (52-54) which are produced at high levels after exposure to UVR. The major and most damaging reactive oxygen species, the hydroxyl radical, results from Fenton/Haber-Weiss reaction with hydrogen peroxide (55) and can be scavenged by melatonin, which is consecutively transformed to an indolyl cation radical and, in the presence of O₂⁻, to AFMK (45). Also, the hydrogen peroxide itself can be directly scavenged by melatonin, which is then transformed into melatonin dioxetane and thereafter to AFMK (56). Both mechanisms appear to be relevant for AFMK formation in our study. Additionally, melatonin conversion to AFMK can involve oxidization by phorbol myristate acetate (PMA) and activation by lipopolysacharides (LPS) in leukocytes (47). Apart from those nonenzymatic mechanisms, AFMK production may be triggeed by cleavage of the melatonin pyrrole ring by indoleamine 2,3-dioxygenase (45), a major catabolic pathway of melatonin in tissues (57). Further metabolism of AFMK by arylamine formamidase leads to formation of N¹-acetyl-5-methoxykynuramine (AMK) (52). In leukocytes, melatonin is oxidized by myeloperoxidase and oxyferrylhemoglobin to AFMK (58, 59) that is further degraded to AMK by catalase (45). Recent studies have shown that AFMK can be also produced by oxidation of melatonin through mitochondrial cytochrome c (48).

The second major melatonin product detected post UVR exposure was 2-hydroxymelatonin previously identified only in Fenton-type OH-generating systems (60) or in reaction with hypochlorous acid (61). The cyclic form of 2-hydroxymelatonin has been detected in the jugular blood in the rat (62) and in human or rat urine after chloroform extraction (63) accounting for 5% of the urinary metabolites of melatonin. Since the levels of 6-hydroxymelatonin in the urine as well as in keratinocytes are much higher than those of 2-hydroxymelatonin, it can be concluded that 6-hydroxymelatonin could be a major product when melatonin undergoes enzymatic metabolism, whereas 2-hydroxymelatonin would predominate during chemical reaction induced by UVR-related oxygen based radicals (60) or by the combination of ROS with enzymes such as cytochrome c (48). The present invention establishes 2-hydroxymelatonin to be a major intermediate between melatonin and AFMK following UVR exposure.

Product 3 was identified as 4-hydroxymelatonin. Interestingly, this product peaked shortly after 2-hydroxymelatonin when detected by HPLC, whereas the order of peak appearance in LC-MS was vice versa. This could be explained by use of different mobile phase for HPLC (neutral medium) and mass spectrometry (acid medium). Both products are generated after hydroxylation at carbon C2 and C4, and C2 seems to be the most favourable site for primary hydroxyl radical addition (64). The lower detection level of 2-hydroxymelatonin in LC-MS, however, may be explained by fast transformation of this product to 2,3-hydroxymelatonin and AFMK.

Previous studies on UVR-induced degradation of melatonin used light emissions in the UV-VIS range (wavelength 300-575 nm), with maximum emission in the UVA range at 365 nm and at 565 nm in the visible light range (44). In the present invention, the UV source emitted primarely in the UVB wavelength (280-320 nm) while a minor fraction in the UVA range (320-340 nm). Both wavelengths are important in cutaneous biology, since UVB causes the severe harmful effects in the epidermis, as represented by direct DNA damage in proliferating keratinocytes (65, 66) and generation of hydrogen peroxide by direct photochemistry, which leads to production of hydroxyl radicals by Fenton/Haber-Weiss reaction (55). Hydroxyl radicals react with melatonin at the carbon positions 2, 3, 4 and 6 (13) to build hydroxymelatonin molecules of which we could identify three (2-, 4- and 6-hydroxymelatonin). UVA reaches deeper layers in the dermis causing fiber shrinkage and elastosis associated with skin aging (67). These processes are the major targets for the protective effects of melatonin, a strong radical scavenger, especially for the hydroxyl radical (15). Therefore, the same specific wavelengths were used to investigate the impact of UVR on melatonin itself. Of the resulting products, some have effects potentially protective: AFMK for example is known to be a strong radical scavenger, thus protecting against free radical formation, lipid peroxidation and oxidative DNA damage (44, 45) and building an antioxidative cascade with melatonin and other melatonin metabolites (13). The increased formation of AFMK under progressively higher doses of UVR would therefore support the use of melatonin substrate in topically applied sun protective preparations, which can penetrate and build a depot in the upper layers of the skin (38). As a result, the organ could remain in an equilibrium between damaging effects of UVR and the protective effects of the UVR-induced increase of melatonin metabolites. This novel cutaneous defense mechanism may be defined as a melatoninergic antioxidative system (MAS).

Kinetic studies on the generation of photoproducts of melatonin in supernatants after UV exposure support their dual origin for cutaneous melatonin; thus, the progressive increase in 2-hydroxymelatonin and 4-hydroxymelatonin over the six-hour period may be explained not only by oxidation of melatonin in suspension, but also by metabolization of intracellular melatonin stores with metabolite production and subsequent release into the extracellular compartment. Of further interest is the late decrease in AFMK at 3 to 6 hrs after UVR exposure that may be explained by additional metabolism of AFMK to AMK by arylamine formamidase. AMK, however, could not be detected, most likely due to very low levels. In a study by Silva et al. (47) the levels of AMK in activated leukocytes were 5 to 10% of AFMK since AMK, in contrast to AFMK, is easily oxidized.

To confirm the keratinocyte related production of 2-hydroxymelatonin and AFMK induced by UVR, the present invention, assayed for the products in cell lysates of keratinocytes, and indeed found 2-hydroxymelatonin and AFMK. Also in analogy to the findings in the cell-free environment and supernatants, AFMK was the predominant metabolite. These observations indicate that the source of AFMK is intracellular melatonin. UVR-induced AFMK production was detected in both, supernatants and cell lysates of keratinocytes not preincubated with melatonin; detectable intracellular melatonin levels were also evident. A melatonin source for AFMK production was further confirmed by the observation of still higher AFMK levels in supernatants and lysates of cells that had been preincubated with high concentration of melatonin (10−3 M). Since the present invention detects melatonin in cell lysates, this demonstrates that melatonin is consumed following UV irradiation. This observation was consistent in supernatants and cell lysates. Also, as expected, the intracellular melatonin levels were higher after preincubation with exogenous melatonin.

Even though melatonin is highly lipophilic and assumed to penetrate easily through lipid membranes (68), the uptake of melatonin into the cell was only 0.125% of applied levels. This low percentage is in agreement with data of Nickel and Wohlrab (69) who found melatonin uptake within the same range (0.097%) in HaCaT keratinocytes. The slight difference found between the two studies might be due to measurement of only the added tracer ([3H]-labeled melatonin) by Nickel and Wohlrab, while measurements included the endogenous pool plus the melatonin taken up from supernatants. Additional factors influencing melatonin uptake by HaCaT keratinocytes may be differences in culture conditions. In the present invention, keratinocytes were cultured in media containing 10% fetal bovine serum, which may increase cellular metabolic activity and uptake of melatonin as compared to keratinocytes cultured in media with lower serum content or serum of different origin. The present invention therefore safely concludes that for HaCaT keratinocytes grown under the described conditions, the addition of melatonin at 10−3 M will lead to intracellular melatonin levels at the concentration of approximately 10−6 M. These intracellular levels detected in HaCaT keratinocytes are still considerably higher than the levels of melatonin in human plasma, which gives another example confirming that melatonin is not in an equilibrium within an organism and that it can show considerable differences in its levels dependent on the compartment in which it is measured. In liquid compartments such as the bile, the bone marrow or cerebrospinal fluid (CSF), melatonin concentrations have been shown to be by orders of magnitude higher than in the plasma (5, 70-72). Also in cells, endogenous melatonin levels can differ significantly from the plasma when the cell populations have production rates that meet their requirements, e.g. in the gastrointestinal tract (3, 73), blood cells (6, 74) and retina (18, 75). Physiological vs. pharmacological levels of melatonin had been recently discussed (18), and it has been suggested that the physiological level of melatonin has to be defined at the local level, dependent on the specific cell, fluid or organelle type (76). In this regard, the present invention, provides the first measurements of intracellular melatonin levels in HaCaT keratinocytes. Both, the presence of a functionally active melatoninergic system in the skin (9, 10, 19, 26, 77) and the detection of melatonin production in human and murine hair follicles (78) support the findings of the instant invention. Previous studies have shown, that melatonin at the same concentration as used in the present study (10−3 M) protects cultured HaCaT keratinocytes against UVR-induced damage (37).

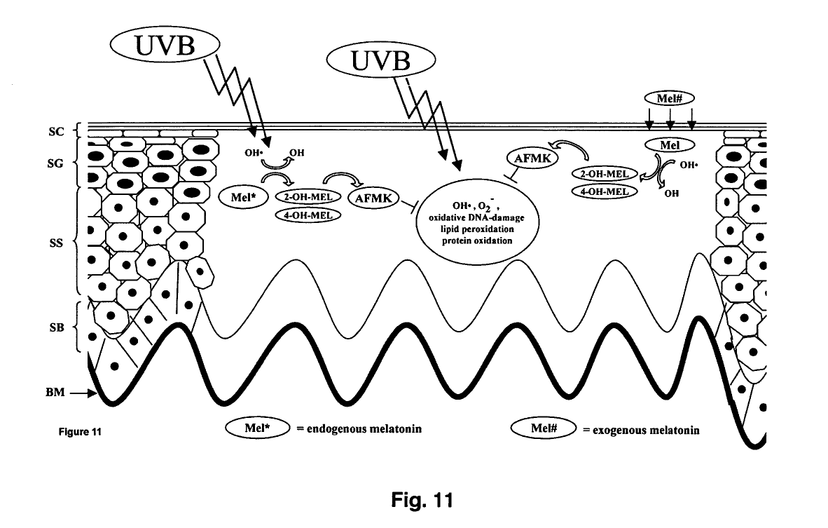

Thus, the present invention demonstrates that, within 24 hrs, keratinocytes metabolize melatonin to AFMK and 6-hydroxymelatonin as major products, with concomitant increase of 2-hydroxymelatonin, the intermediate between melatonin and AFMK, while melatonin consumption is detected. Most importantly, this process can be directly activated by UVR. Thus, melatonin may play an important role in cutaneous biology by protecting the skin against solar radiation. Moreover, generation of the UV-induced melatonin metabolites which are strong antioxidants and therefore protective substances themselves defines a novel melatoninergic antioxidative system (MAS) of the skin (FIG. 11). Finally, the combination of endogenous melatonin with externally applied melatonin may successfully counteract the multiple processes of skin damage induced by UVR.

Further, the instant invention demonstrates that melatonin attenuates UVR induced changes in keratinocytes morphology. This is accompanied by melatonin inhibition of UV-induced mitochondrial apoptotic pathway as reflected by attenuation of mitochondrial membrane potential reduction, reduced activation of initiator caspase 9 and effector caspases 3 and 7, and reduced PARP activation.

Apoptotic signaling events and their prevention by melatonin treatment at the UV dose of 50 mJ/cm², were also investigated since previous studies had demonstrated that this UV-dose caused considerable reduction of cell viability, clonogenic cell growth and DNA-fragmentation, which was significantly prevented by melatonin (48). Similarly other studies showed that 50 mJ/cm² but not 15 mJ/cm² was the most relevant UV-dose for apoptosis induction with consecutive activation of casp-9, casp-3, casp-7 and PARP (68, 69) and the same UV-dose led to cytochrome c release followed by activation of casp-3, -8 and -9 (70). Sitailo et al. used similar UV-source emission spectrum (UVB ˜65%; UVA ˜34%) and investigated caspase activation under a slightly lower UV-dose of 30 mJ/cm² in normal human keratinocytes and HaCaT keratinocytes (58). Casp-3 was found to be activated to the greatest extent, and the activation of casp-9 was stronger than casp-8, indicating that the intrinsic (mitochondrial) pathway would represent a major determinant in UV-induced apoptosis in keratinocytes. Some authors assumed that casp-8 activation is a bystander effect of the mitochondrial apoptotic pathway and that casp-8 is activated downstream of casp-9 and casp-3 activation (50). More recent studies, however, showed that activation of casp-8 is an UVB specific event and independent from casp-9 and casp-3.

The instant invention investigates different regulatory points of apoptosis for the first time under the treatment with melatonin. The instant invention demonstrates activation of caspase 8 at 24 hrs after UV-irradiation; however, melatonin had no effect on its reduction. This might be explained by the fact that this pathway is not activated by formation of reactive oxygen species (ROS) and accordingly cannot be counteracted by the antioxidant melatonin. In contrast, the mitochondrial pathway, activated by mROS formation, was strongly influenced by melatonin. Specifically, mitochondrial membrane potential was reduced upon exposure to UV irradiation and melatonin prevented membrane potential reduction at the concentration of 10⁻³, 10⁻⁴ and 10⁻⁶ M, comparable to earlier observed antiapoptotic effects in HaCaT keratinocytes (48). Consequently, the mitochondrial pathway initiator caspase 9 was activated, followed by activation of the downstream effector caspases casp-3 and casp-7. Casp-3, hereby, seems to be the most relevant, since casp-3 deficient mouse embryonic stem cells cannot execute UV-induced apoptosis (71, 72). In our study, UV-induced activation of both effector caspases were reduced in keratinocytes preincubated with melatonin, which might be a consequence of reduced activation of up-stream caspases or direct inhibition of effector caspase activation by melatonin. Support for the latter is given by the observation that melatonin can prevent neuronal death in mouse brain ischemia by direct inhibition of casp-3 (73) and, that also casp-3 is directly reduced by melatonin in aflatoxin B1 treated liver cells (74). There was also a tendency of earlier activation of casp-9 compared to casp-3 and 7, underlining the chronologic cascade of the caspase related apoptotic activation sequence.

In contrast, PARP was activated at 24 hrs representing an early event of UV-induced apoptosis. PARP is a Zn-finger nuclear protein, activated by single-strand DNA breaks, reactive oxygen species and disruption of mitochondrial membrane potential (61-65). PAR, the resulting product after UV-induced PARP activation, was identified as an early marker of apoptosis that is positive in UV-exposed HaCaT keratinocytes at 24 hrs post UV irradiation (62). This is in agreement with our result., showing a stronger expression at 24 hrs compared to 48 hrs. PARP is needed for DNA repair upon ROS-induced damage (75), and reduction of PARP parallels with reduction of single-strand DNA-breaks (65). Thus, it can be concluded that reduction of PARP activation by melatonin consequently reduces DNA damage. Saldeen et al. have shown that PARP cleavage can also be directly induced by disruption of mitochondrial membrane potential (61). Therefore, the reduction of PARP activation by melatonin may be explained by reduced mitochondrial damage, as shown in the present study, and/or by ROS reduction through intramitochondrial/cytosolic melatonin, since melatonin is a potent radical scavenger (29, 46). Additionally, we showed in earlier studies that UV-induced DNA-fragmentation in keratinocytes is indeed successfully reduced under treatment with melatonin (48). Thus, the diminished mitochondrial membrane potential reduction, followed by reduced activation of caspase 3, 7 and 9 together with reduced degree of DNA damage reflected by attenuated PARP cleavage leads to the survival of a “healthy” cell population in UV-irradiated keratinocytes treated with melatonin.

The instant invention also investigated mitochondrial membrane potential reduction under UVR exposure. Oxidative stress and concomitant formation of mitochondrial reactive oxygen species (mROS) leads to calcium influx into the mitochondria with consecutive opening of the mitochondrial permeability transition pore (MPTP) (77) and depolarization of the mitochondrial membrane potential (56), the end result of UV-induced mitochondrial damage as found in our study. Melatonin has been shown to act against apoptosis on mitochondrial level by direct inhibition of the MPTP, a newly identified mechanism responsible for anti-apoptotic effects of melatonin (77). Melatonin interacts in this pathway by reduction of mROS and calcium as well as by inhibition of the opening of the MPTP as shown in rat brain astrocytes (56), mouse striatal neurons (77) and rat cerebellar granule neurons (78). The instant invention also demonstrates that in skin keratinocytes melatonin preserved mitochondrial membrane potential from UV-induced reduction. Since mitochondrial damage is a very sensitive and early event in UV-induced apoptosis, this fact underlines—additional to the observed PARP inhibition by melatonin—the strong and pluripotent protective effects of melatonin which intercalates at the two main apoptotic pathways in UV-exposed keratinocytes.

To conclude, the present invention demonstrates for the first time in keratinocytes, the cell population in the skin to which UV radiation is most relevant, that melatonin is able to prevent execution of apoptosis pathways induced by 50 mJ/cm² UVB at the most relevant cellular levels of apoptosis. Melatonin maintains the mitochondrial membrane potential, a key-event in early apoptosis development, inhibiting the consecutive intrinsic apoptotic pathway on the caspases level and independently reduces PARP activation, a sensitive marker for DNA damage.

Hence, in one embodiment of the present invention, there is provided a method of protecting the skin from ultra-violet radiation comprising of applying topically an effective dose of a formulation comprising melatonin; wherein said application protects the skin from the damaging effects of ultra-violet radiation. Specifically, protection is due to scavenging of UVB-induced reactive oxygen species by melatonin. Further, the protection is due to generation of melatonin photoproducts. Specifically, the melatonin photoproducts are 6-hydroxymelatonin, 2-hydroxymelatonin, 4-hydroxymelatonin and N¹-acetyl-N²-formyl-5-methoxy-kynuramine. Additionally, the metabolites generated due to metabolism of melatonin protect the skin against lipid peroxidation, protein oxidation and DNA damage. Specifically, the damaging effects of UVR include photoaging and cancer.

In another embodiment, there is provided a method of preventing or reducing photodamage to the skin of a subject comprising administering a composition comprising melatonin to the subject in an amount effective to inhibit UVB-induced apoptosis of skin cells exposed to ultra violet radiation. Further, the inhibition of the UVB-induced apoptosis comprises attenuation of the mitochondrial membrane potential reduction, reduced activation of caspase 9, 3 and 7, and reduced PARP activation. Moreover, the reduced PARP activation by melatonin is due to the attenuation of mitochondrial damage and/or by reduction of the reactive oxygen species. In general, the composition is a topical composition. Specifically, the topical composition is in a form selected from the group consisting of a cream, gel, salve, lotion or spray.

In yet, another embodiment there is provided a method of retarding development of skin cancer in an individual at risk comprising topical application of an effective dose of a formulation comprising melatonin as the single active ingredient; wherein said application retards development of skin cancer in said individual at risk. In general, the protection is due to generation of melatonin photoproducts. Specifically, the melatonin photoproducts are 6-hydroxymelatonin, 2-hydroxymelatonin, 4-hydroxymelatonin and AFMK. In general, melatonin photoproducts are generated due to metabolism of the exogenously applied melatonin. Additionally, melatonin metabolites scavenge UVB induced reactive oxygen species (ROS) and/or inhibit UVB-induced apoptosis of skin cells exposed to ultra violet radiation. Specifically, the UVB-induced apoptosis is mediated by the mitochondrial apoptotic pathway in skin cells. Additionally, the inhibition of the UVB-induced apoptosis comprises attenuation of the mitochondrial membrane potential reduction, reduced activation of caspase 9, 3 and 7, and reduced PARP activation. In general, the composition is a topical composition. Specifically, the topical composition is in a form selected from the group consisting of a cream, gel, salve, lotion or spray. In general, the skin cancer is from the group consisting of basal cell carcinoma, squamous cell carcinoma or melanoma.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Cell Culture

HaCaT keratinocytes were cultivated in Dulbecco's Modified Eagle Medium (DMEM) supplemented with glucose, L-glutamine, pyridoxine hydrochloride (Gibco, Invitrogen Life Technologies Carlsbad, Calif.), 10% fetal bovine serum (Mediatech Inc., Herndon, Va.) and 1% penicillin/streptomycin/amphotericin antibiotic solution (Sigma Chemical Co., St. Louis, Mo.). Cells were trypsinized from culture flasks and seeded in 10 cm petri dishes (Corning Inc., Corning, N.Y.) at a density of 10⁶ cells/dish and incubated overnight. The next day, after confluence of 80-90% was reached, cells were washed once with PBS to remove remnants of media, and incubation with melatonin in PBS was performed for 30 min. Parallel control dishes were incubated with PBS without melatonin. After incubation the Petri dishes were irradiated from below with UVR. For the investigation of melatonin uptake and metabolism, keratinocytes were incubated with melatonin for 30 min or 24 hrs; endogenous melatonin production in keratinocytes was investigated in the absence of previous melatonin incubation. For cell-free experiments, melatonin in pure PBS was irradiated with UVR.

### EXAMPLE 2

Melatonin and HPLC Standards

Melatonin was purchased from Sigma Chemical Co. (St. Louis, Mo.) and dissolved in absolute ethanol and further diluted with PBS (final concentration of ethanol <0.2%). Melatonin solution was added to cells at concentrations of 10⁻³ and 10⁻⁶ M for a 30 min or 24 hrs incubation. Internal standards for HPLC were dissolved in absolute ethanol. These included AFMK, 6-hydroxymelatonin (6-OH-Mel), 5-methoxytryptamine (5-MT), 5-methoxy-3-indol acetic acid (5-MIAA) and 5-methoxytryptophol (5-MTphol). All reagents, except for AFMK, were purchased from Sigma Chemical Co. (St. Louis, Mo.). AFMK was produced as described previously (48). AFMK was then purified by HPLC and its identity confirmed by UV spectra (at λmax=231, 262 and 342 nm) and verified by the mass spectrometry (findings of molecular ion [M+H]+ at m/z 265 and fragment ions at m/z 237 ([(M-N-acetyl)+H]+) and m/z 178 ([(M-(N-acetyl+N-formyl))+H]+).

### EXAMPLE 3

UV Irradiation

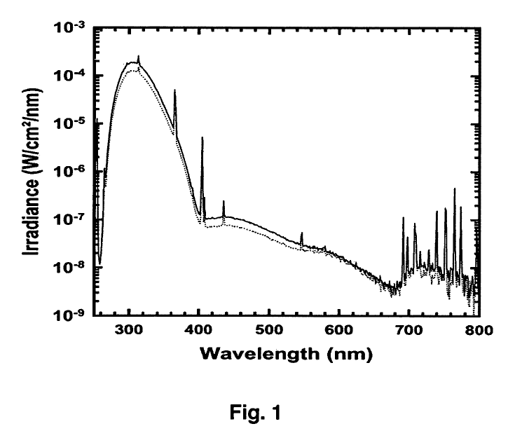

Irradiation experiments were performed with a Biorad UV transluminator 2000 (Bio-RAD Laboratories, Hercules, Calif.), calibrated as described previously (37). Briefly, a scanning double monochromator spectroradiometer (Model OL 754, Optronic Laboratories, Orlando, Fla.) was used to scan emission of wavelengths from 250 to 800 nm at 1 nm increments. The spectroradiometer had been calibrated with an NIST traceable tungsten-halogen spectral irradiance standard (Model 75-10E, Optronic Laboratories, Orlando, Fla.) with a precision current source (Model 65, Optronic Laboratories, Orlando, Fla.). An additional calibration module (Model 752-150, Optronic Laboratories, Orlando, Fla.) measured both photometric gain and wavelength accuracy. Wavelength calibration and gain were established or verified before each experimental use. The UV source emission (shown in FIG. 1) consisted primarily of UVB light (wavelength 280-320 nm; 60%), with minor output in the UVA (320-400 nm) and UVC (120-280 nm) range (˜30% and ˜10%, respectively). Melatonin photoproducts generation was performed after irradiation at the UV doses of 25, 50, 75 or 100 mJ/cm², as indicated in the figures.

### EXAMPLE 4

### Collection and Extraction of Samples

Samples of melatonin prepared in a cell-free environment were incubated for 30 min prior to irradiation in parallel to melatonin-exposed keratinocytes and frozen at −80° C. immediately after irradiation. Supernatants from HaCaT keratinocytes preincubated with melatonin and submitted to UVR were collected at 10 min, 40 min, 190 min and 370 min after UVR exposure and frozen at −80° C. pending analysis. Cell pellets contained in 100 μl PBS were collected by trypsinization and centrifugation, and were also frozen (−80° C.) until further processing. Supernatants and cell-free melatonin-PBS samples (25 ml) were extracted twice with 3 volumes of methylene chloride and the pooled sample dried in a vacuum evaporator (Buchi Labortechnik AG, Flawil, Switzerland). Cell pellets were sonicated (Vibra Cell; Sonics and Materials, Danbury, Conn.) in 5 ml of ice-cold PBS and were subjected to organic extraction from 25 ml PBS, as outlined above. Sample processing was conducted entirely under conditions of low ambient light and extracts were frozen at −80° C. pending HPLC analysis.

### EXAMPLE 5

High Performance Liquid Chromatography Analysis

Cell extracts were briefly sonicated in 50 μl absolute ethanol in a Branson 5200 waterbath at ambient temperature (Branson Ultrasonics Corporation, Danbury, Conn.). The re-suspended contents were transferred to 100 μl borosilicate glass vials (JP Cobert and Associates, St. Louis, Mo.) in a refrigerated (5° C.) automated injector. HPLC analysis (all equipment, Waters Associates, Milford, Mass.) of 20 μl aliquots was accomplished with a C18 Nova-pak™ reverse-phase column (4 μm particle size; 10 cm×5 mm id) using a gradient (5%-15% over 40 minutes) of HPLC-grade acetonitrile (Fisher Scientific, Fairlawn, N.J.) in phosphate buffer (0.01M; pH 7.2) at 1.0 ml/min. Column eluate was monitored by Model 2487 UV detector (275 nm) and Model 991 photodiode array detectors and the data stored electronically for subsequent interpretation. Melatonin and related standards (in 5-20 μl absolute ethanol) were analyzed in an identical manner and identification of sample peaks made by correspondence to retention time, and where possible, absorption spectrum.

### EXAMPLE 6

LC-MS Analysis

Aliquots (20 ml) of samples from the cell suspension and cell-free experiments were separated on an LC-MS QP8000a (Shimadzu, Japan) equipped with diode array and single quadrupole mass-spectrometric detectors. The separation system consisted of a Restec Allure C18 reverse-phase column (150×4.6 mm; 5 μm particle size; and 60 A pore size) with mobile phase consisting of 25% acetonitrile and 0.1% acetic acid. Elution was carried out isocratically at flow rate of 0.75 ml/min and temperature of 40° C. The eluent was routed to the mass-spectrometric electrospray interface (ESI) set in positive mode and using nitrogen as the nebulizing gas. Mass-spectrometry parameters were as follows: nebulizer gas flow rate, 4.5 l/min; electrospray voltage, 4.5 kV; and curved desolvation line (CDL) heater temperature, 250° C. The selected ion monitoring (SIM) mode was used to detect ions with m/z=249 (monohydroxymelatonin); m/z=265 (dihydroxymelatonin and AFMK); m/z=237 (AMK); and m/z=233 (melatonin). System control and data acquisition were performed with the LC-MS workstation Class-8000 software (Shimadzu, Japan).

### EXAMPLE 7

Melatonin Treatment

Melatonin (Sigma Chemical Co., St. Louis, Mo.) was dissolved in ethanol, further diluted with PBS (final concentration of ethanol<0.2%) and added to medium to achieve test concentrations. Cell media from overnight incubation was removed and replaced with fresh media containing melatonin at the concentration of 10⁻³ M, the maximum effective concentration identified in previous studies (45, 46). After incubation with melatonin for 12 or 24 hours before irradiation, melatonin containing media were removed, cells were washed once with PBS to remove remnants of media and melatonin, and PBS was added another time to keep cells covered by PBS during UVR exposure. Irradiation of the Petri dishes was performed with UVR from below, and after irradiation PBS was replaced by fresh culture media for 24 or 48 hrs. Paraely, cells were incubated with or without melatonin, but not subjected to irradiation to exclude effects of melatonin alone.

### EXAMPLE 8

Morphology Analysis

Digital pictures from six to ten randomly chosen fields per Petri dish for each experimental condition were acquired at different time points after UV exposure (0, 24 and 48 hrs) with a NIKON Eclipse TE300 microscope (Melville, N.Y.). At 24 and 48 hrs after UV exposure, pictures of detached cells were first taken, then detached cells were removed and another set of pictures was acquired to assess the degree of confluency of cells which were still attached to the bottom of the culture dish. Pictures were recorded and analyzed with MetaVue software. Then, keratinocytes were harvested from Petri dishes by trypsinization, washed three times with ice-cold PBS and frozen in −80° C. until further processing.

### EXAMPLE 9

Measurement of Mitochondrial Membrane Potential (ΔΨ) Using JC-1

Mitochondrial inner membrane potential (ΔΨ) in immortalized HaCaT keratinocytes was measured by using confocal microscopy with 5,5′,6,6′tetrachloro-1,1′,3,3′-tetraethylbenzimidazol-carbocyanine iodide (JC-1) (Molecular Probes, Carlsbad, Calif.) (66). JC-1 selectively enters mitochondria and aggregates when the membrane potential values exceed 80-100 mV, causing a shift in fluorescence from 530 nm (green) to 590 nm (red). For experiments, human HaCaT keratinocytes were seeded in Lab-Tek II 8-well chambered coverglass (Nalge Nunc, Inc., Naperville, Ill.) and grown until 90-100% of confluence. Culture media were removed and cells washed with PBS and then incubated for 30 or 120 min with melatonin at concentration. Stock solution of melatonin was dissolved in DMEM and added to the culture media. After incubation, cells were washed twice with PBS and subjected to irradiation with UVB at the dose of 50 mJ/cm² or incubated with serum free medium containing 1 mM H₂O₂ for 1 h. After irradiation cells were incubated in DMEM containing 5% FBS and supplemented with JC-1 (2.5 μg/ml) for 30 min at 37° C. Then, cells were washed with serum containing medium and slides were observed with laser scanning confocal fluorescent microscope (LSM 510, Carl Zeiss GmbH, Jena, Germany) equipped with Plan-Neofluor oil immersion 40× objective with suitable filter setup. Images were acquired from three to six randomly chosen fields for each experimental condition showing nuclear cross-section. Green and red channels were merged and ratio of red to green channel was shown in blue.

### EXAMPLE 10

Immunoblot

Cell pellets were mixed with lysis buffer (PBS containing Triton X 100 0.2% and 1 μl protease inhibitor per 100 μl buffer) and left on ice for 30 min. After centrifugation for 5 min at 9000 g the supernatant was taken for protein determination performed by BCA Protein assay kit (Pierce, Rockford, Ill.). Cell lysates were aliquoted in four samples per condition for repeated immunoblotting or incubation with different antibodies. For each immunoblot, lysates containing 50 μg protein were mixed with loading-buffer, boiled for 5 min at 95° C. and then separated on a 12 or 15% SDS-PAGE gel (PAGEr Duramide Precast Gel, Cambrex Bio Science, Rockland, Me.). Parellely, biotinylated protein ladder (Cell Signaling Technology, Inc. Danvers, Mass.) was used as a marker. Proteins were then blotted onto Immobilon-P polyvinylidene fluoride (PVDF) membrane (Millipore Corp, Bedford, Mass.). After blotting, membranes were blocked with 5% non-fat dry milk in TBS-Tween 0.1% for 1 hr with gentle shaking and then washed three times with TBS-Tween 20 alone. Next, membranes were incubated with specific primary antibody in 5% non-fat dry milk overnight at 4° C. Antibody used were as follow: rabbit anti-caspase 3, 7 and 9 antibody (1:1000) and against the specific cleaved forms of caspases 3, 7 and 9 (1:500); rabbit PARP and cleaved PARP antibody (1:1000) Caspase 8 was detected with mouse anti-caspase 8 antibody (1:1000). All antibodies were purchased from Cell Signaling Technology, Inc., (Danvers, Mass.). Caspases-8 is a 57 kDa protein with cleaved forms at 43 and 18 kDa, casp-9 (47 kDa) with cleaved forms at 35 and 17 kDa, casp-3 (35 kDa) with cleaved forms at 19 and 17 kDa, casp-7 (35 kDa) with cleaved form at 20 kDa and PARP (116 kDa) with cleaved forms at 89 and 24 kDa. After incubation with primary antibodies, membranes were incubated with secondary goat anti-rabbit or anti-mouse HRP-linked IgG antibody (1:2000) in presence of anti-biotin HRP-linked antibody for the protein ladder (1:2000) at room temperature for 1.5 hours. Bands were visualized by SuperSignal West Pico reagents (Pierce Biotechnology, Inc. Rockford, Ill.) and chemiluminescence was analysed by Fluor-S Multi-Imager using Quantity One software (both Biorad Laboratories, Hercules, Calif.). Additionally, membranes were exposed to autoradiography film and developed with photodeveloper and fixer (Kodak, Rochester, N.Y.). Densitometry was performed with Scion Image analysis software (NIH).

### EXAMPLE 11

Statistical Analysis

Bands of immunoblots were evaluated by measurement of density with Scion Image analysis software (NIH). Density values from two or three experiments were taken for calculation of mean values and standard deviation. For statistical analysis of mitochondrial membrane potential, images showing nuclear cross-section were acquired from three to six randomly chosen fields -or each experimental condition. Ration of red/green emission was calculated using ImageJ software (NIH, Bethesda, Minn.) and differences were analysed with Student's t-test and were considered significant when p-value was <0.05.

### EXAMPLE 12

Melatonin Metabolites Induced by UVR in a Cell-Free System

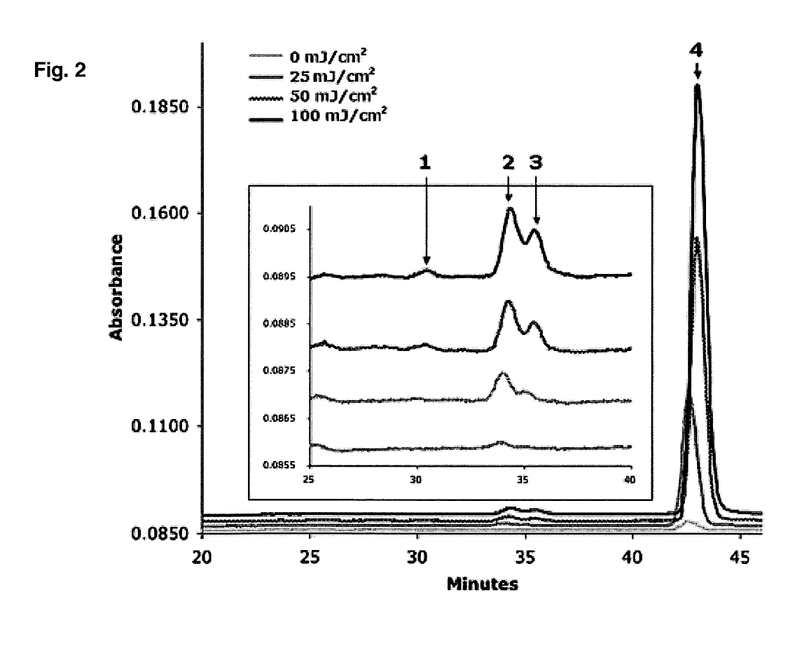

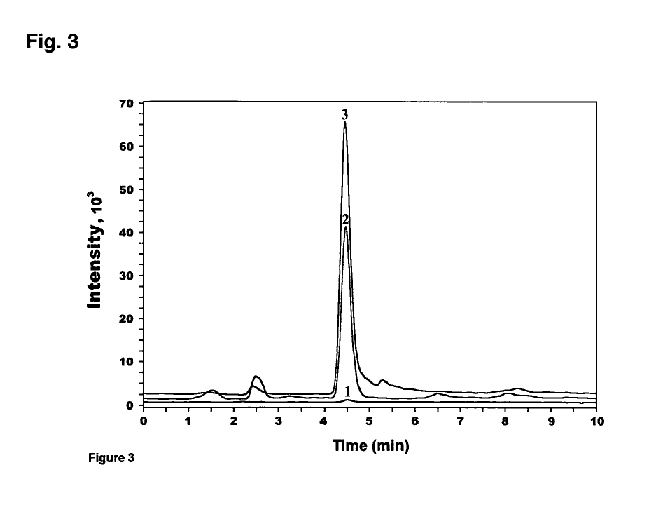

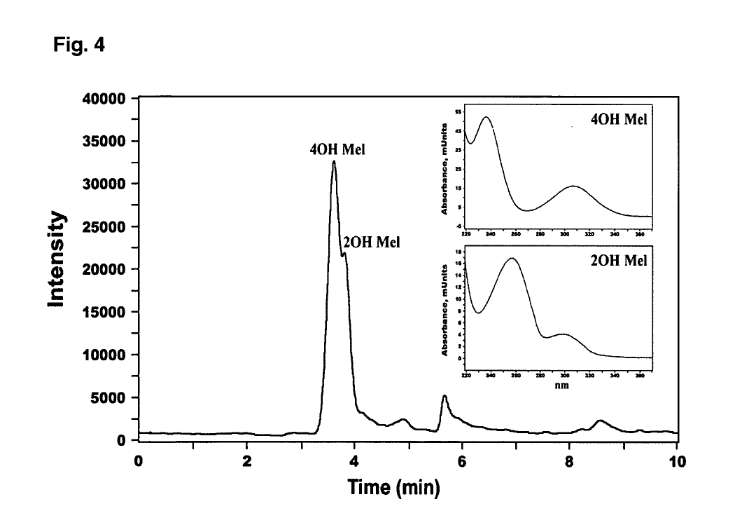

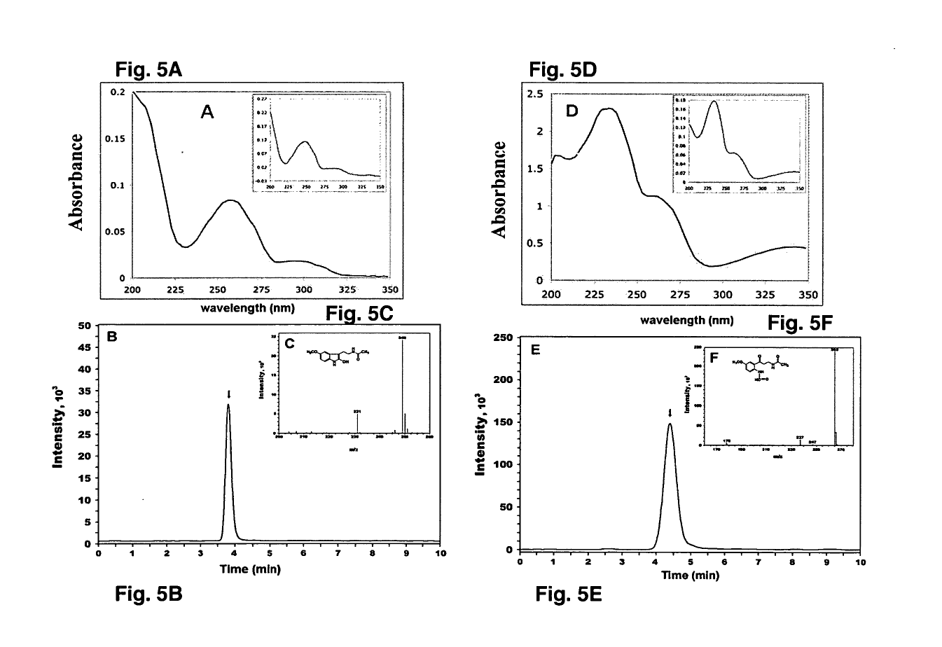

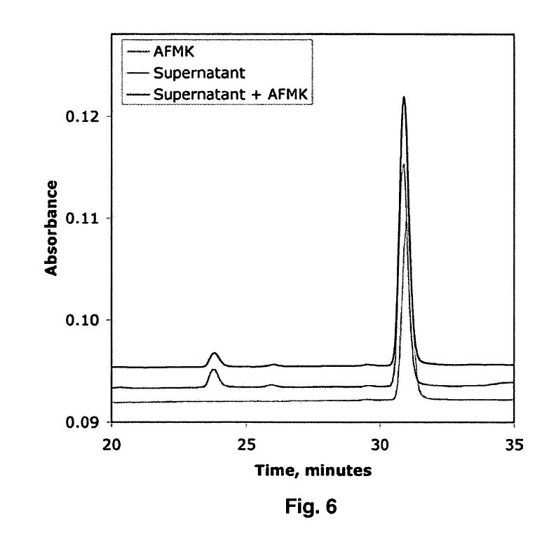

Irradiation of melatonin solution in a cell-free system (melatonin in PBS; UV-doses of 25, 50 and 100 mJ/cm²) generated four compounds detected by HPLC at the retention times (RT) of 30 min (product 1), 34 min (product 2), 35 min (product 3) and 43 min (product 4). Peak areas increased linearly with UV-doses (FIG. 2). Overall, product 4 showed the largest peak, followed by products 2, 3 and 1. In addition, peak size was also dependent on the preincubation concentrations of melatonin with higher concentrations of melatonin (10−3 M) producing larger photoproduct peaks as compared to the peaks from solutions containing melatonin at lower concentration (10−6 M). In the condition of lower preincubation concentration, only the major product 4 was detectable (data not shown). The UV dose-dependent increase of product 4 was further confirmed by LC-MS (FIG. 3). Product identity was determined by comparison with known standards of melatonin metabolites. Matches were found for product 1, 2, 3 and 4. Product 1 was identified as 6-hydroxymelatonin, product 2 as 2-hydroxymelatonin, product 3 as 4-hydroxymelatonin and product 4 as AFMK (FIG. 3-6).

### EXAMPLE 13

Determinants of Post Irradiation Melatonin Metabolism in Keratinocyte Supernatants

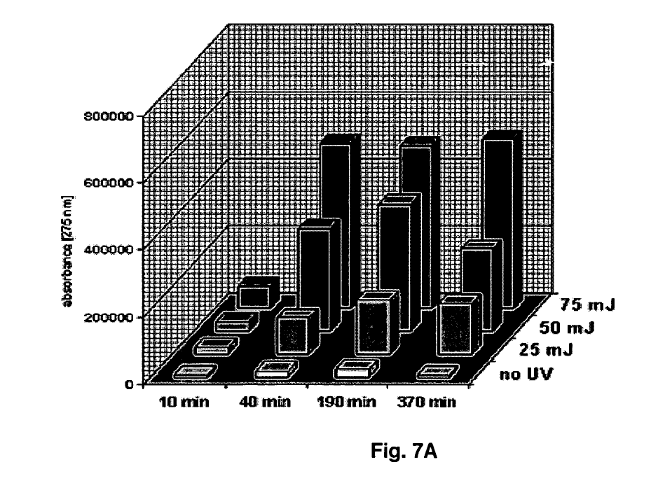

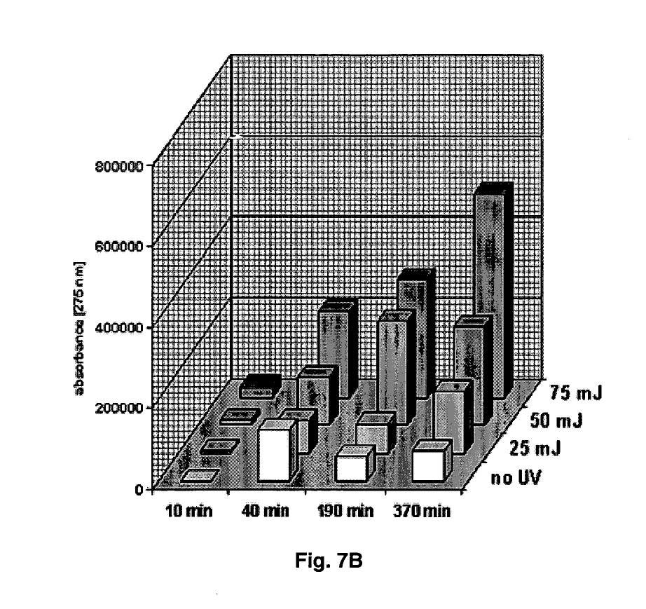

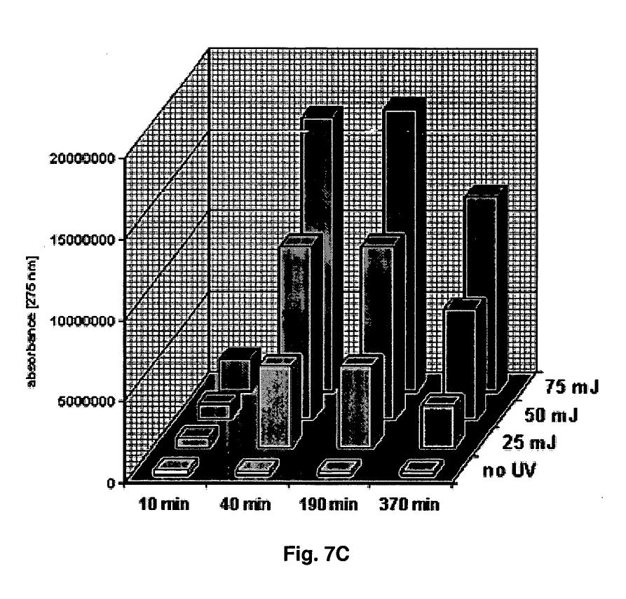

Supernatants of keratinocytes incubated in melatonin at the concentration of 10−3 M produced similar absorption spectra after irradiation with 25, 50 or 75 mJ/cm² with a UV-dose dependent effect. The metabolites detected in the supernatants corresponded to 2-hydroxymelatonin (product 2), 4-hydroxymelatonin (product 3) and AFMK (product 4) with the highest levels seen after irradiation with 75 mJ/cm². There was also a time effect, with larger increases in melatonin photoproducts in supernatants collected at later time points after UV exposure. Thus, metabolite levels were lowest immediately after UV irradiation and increased progressively thereafter. High levels of 2-hydroxymelatonin were detected as early as 40 min after UV exposure and remained elevated at 190 min and 370 min post UVR-exposure (FIG. 7A). Levels of 4-hydroxymelatonin were generally lower than those of 2-hydroxymelatonin, although 4-hydroxymelatonin increased steadily, similar to 2-hydroxymelatonin, from 40 min to 370 min post UV exposure (FIG. 7B). AFMK also showed a time dependent increase with highest levels already reached at 40 min, to remain almost unchanged through 190 min and decreasing at 370 min (FIG. 7C). As in previous experiments with PBS (cell-free system), the overall AFMK levels were considerably higher than those of 2-hydroxymelatonin and 4-hydroxymelatonin.

### EXAMPLE 14

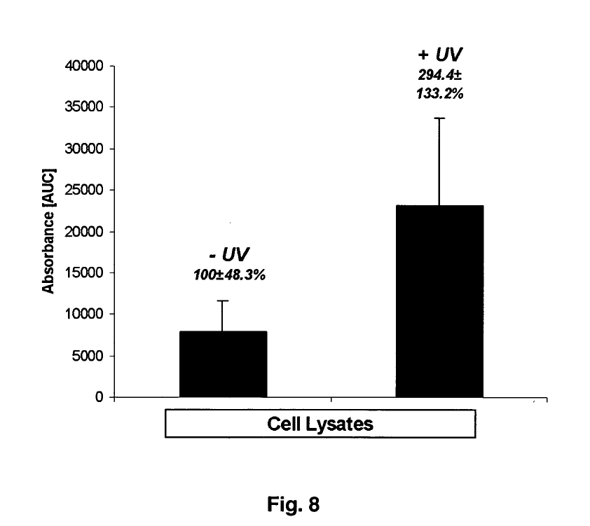

Differential Partition of Melatonin Metabolites

Whereas 2-hydroxymelatonin, 4-hydroxymelatonin and AFMK were identified in keratinocyte supernatants and cell lysates, 2-hydroxymelatonin was present at higher levels in the extracellular compartment, where it displayed strong increases after irradiation with 50 mJ/cm² (data not shown). 2-hydroxymelatonin was nevertheless detectable intracellularly, but at very low levels, while still showing an increase after UVR exposure, as seen in the previous cell-free experiments; the post UVR level was approximately 3-fold greater than the level in nonirradiated samples (FIG. 8). 4-hydroxymelatonin was not detected intracellularly, neither under basal conditions (without UV irradiation) nor after UVR exposure. 4-hydroxymelatonin was however detected in supernatants preincubated with melatonin and exposed to UVR (data not shown).

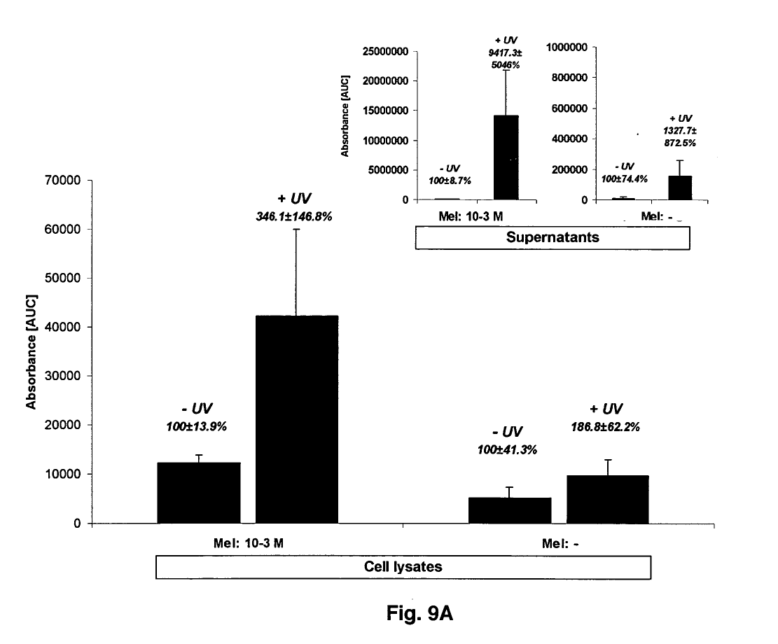

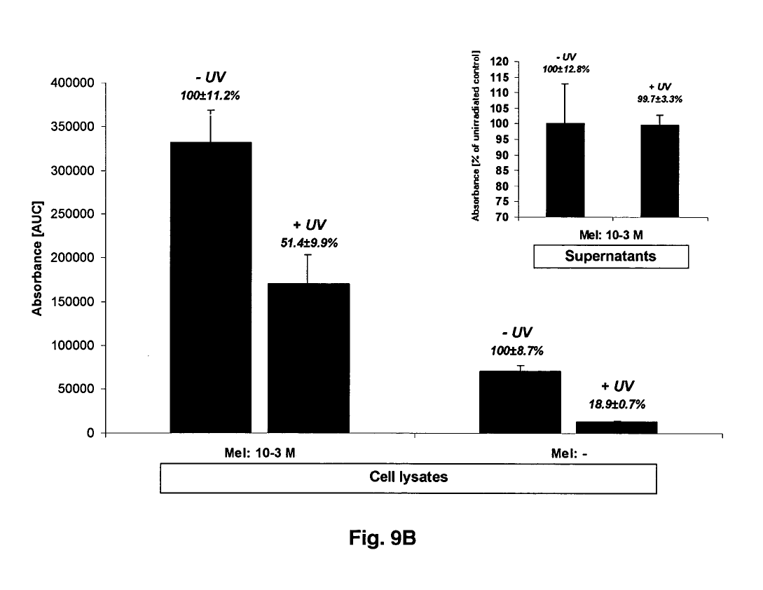

AFMK was detectable in supernatants as well as in cell lysates after melatonin preincubation, almost 100-fold higher in supernatants of irradiated samples (50 mJ/cm²) compared to those non-irradiated (FIG. 9A, upper left inset). Cell lysates generally showed lower levels of AFMK than supernatants, but the UV dependent increase was also observed. AFMK levels in lysates of cells irradiated with UVR were approximately 3.5-fold higher than the levels in nonirradiated lysates (FIG. 9A, left).

Interestingly, AFMK was still detected in supernatants of cells that had not been preincubated with melatonin, albeit at much lower levels. Moreover, the UV-dependent increase resulted in levels almost 13-fold higher as compared to nonirradiated samples (FIG. 9A, upper right inset). AFMK was also detected in native keratinocytes not preincubated with melatonin, although at levels lower than in supernatants. Similar to the observations above, a distinct UV-stimulated increase of 1.9-fold was also evident (FIG. 9A, right).

Melatonin, the main substrate for AFMK production, was predictably detected at high levels in supernatants of samples preincubated with melatonin (10−3 M). These levels decreased slightly after UV exposure (to 99.7% of levels in the nonirradiated melatonin solution) (FIG. 9B, inset). Melatonin was not found in supernatants without melatonin preincubation. In contrast, cell lysates showed detectable levels of melatonin even if the cells had not been incubated with melatonin (FIG. 9B, right). Cell lysates from keratinocytes preincubated with melatonin showed as expected higher melatonin levels than the lysates from cells not preincubated with melatonin (FIG. 9B, left). Nevertheless, with or without melatonin preincubation, cell lysates showed a decrease in melatonin levels after UV exposure (to 51.4% and 18.9% of unirradiated control, respectively) that was reciprocal to the increase of AFMK. Under basal conditions (without UV exposure), the ratio of intra- to extracellular melatonin of samples preincubated with melatonin was approximately 1:800 (0.125%).

### EXAMPLE 15

Kinetics of Melatonin Metabolism in Keratinocytes

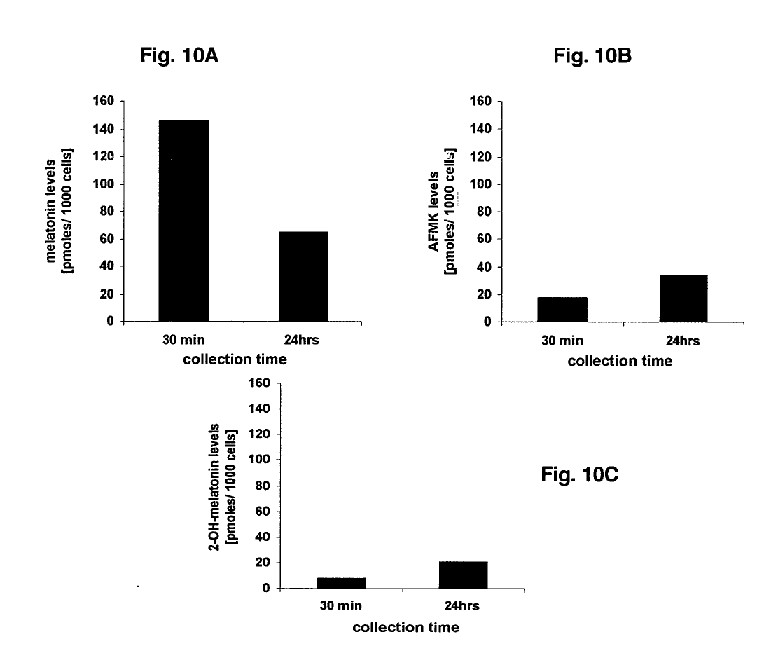

Melatonin and its metabolites AFMK and 2-hydroxymelatonin were detected at low levels in cell lysates of untreated keratinocytes (not preincubated with melatonin). The intracellular level of melatonin in untreated keratinocytes was 146.0 pmoles/1000 cells decreasing to 65.0 pmoles/1000 cells after cultivation for 24 hrs (FIG. 10A). Conversely, AFMK was detected at 17.4 pmoles/1000 under basal conditions, increasing to 33.6 pmoles/1000 cells after 24 hrs (FIG. 10B). Intracellular 2-hydroxymelatonin increased at 24 hrs, although to levels generally lower than those of AFMK and melatonin. The initial concentration of 2-hydroxymelatonin was 7.8 pmoles/1000 cells, this increased to 20.4 pmoles/1000 cells after 24 hrs (FIG. 10C). The metabolite 6-hydroxymelatonin was also detected (53.4 pmoles/1000 cells), but decreased after 24 hrs (16.8 pmoles/1000 cells; data not shown).

### EXAMPLE 16

Melatonin Prevents Cells' Detachment and Blebbing after UV Irradiation

Keratinocytes in non-irradiated condition showed no differences between melatonin treatment and cultivation without melatonin and a continuous increase of confluency over 48 hrs was observed (FIG. 12A). 24 hours after irradiation (50 mJ/cm²) cells showed significant less confluency when compared to non-irradiated cells and cell detachment occurred resulting in empty spaces (ES) (FIG. 12B). In melatonin treated culture dishes, confluency was higher compared to non-treated control and no empty spaces were seen. Additionally, a higher number of detached cells was seen in non-melatonin treated Petri dishes compared to those treated with melatonin. Detached cells showed dysmorphic cell shape, were more swollen and expressed nuclear condensation (NC) in samples not treated with melatonin vs. melatonin treated cells indicative for apoptosis.

### EXAMPLE 17

Mitochondrial Membrane Potential

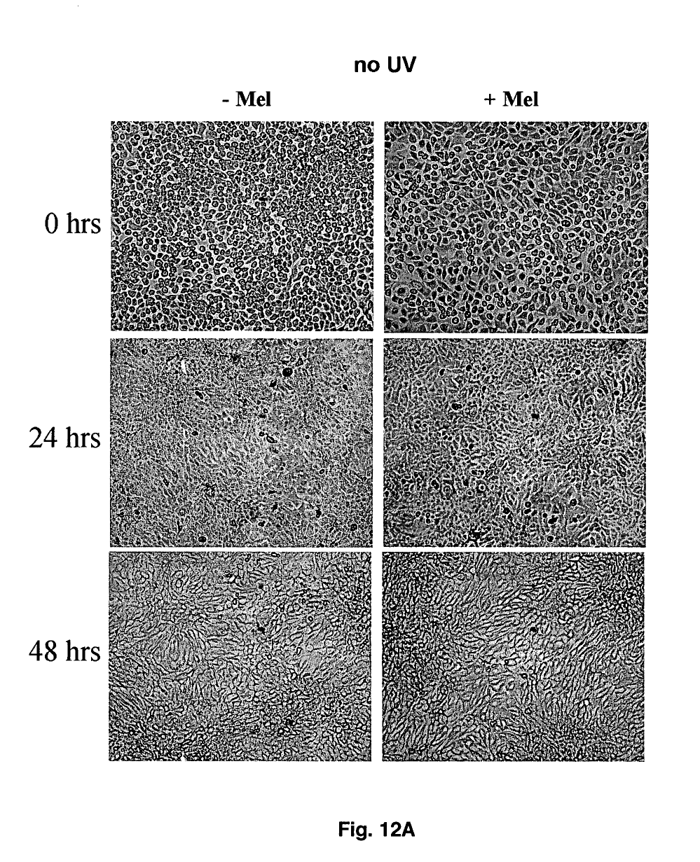

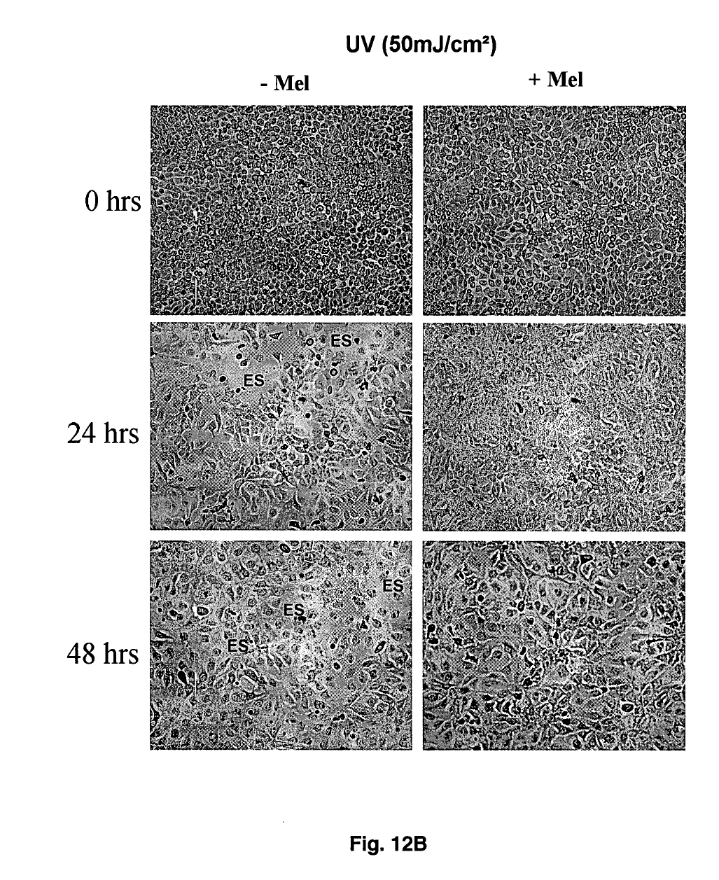

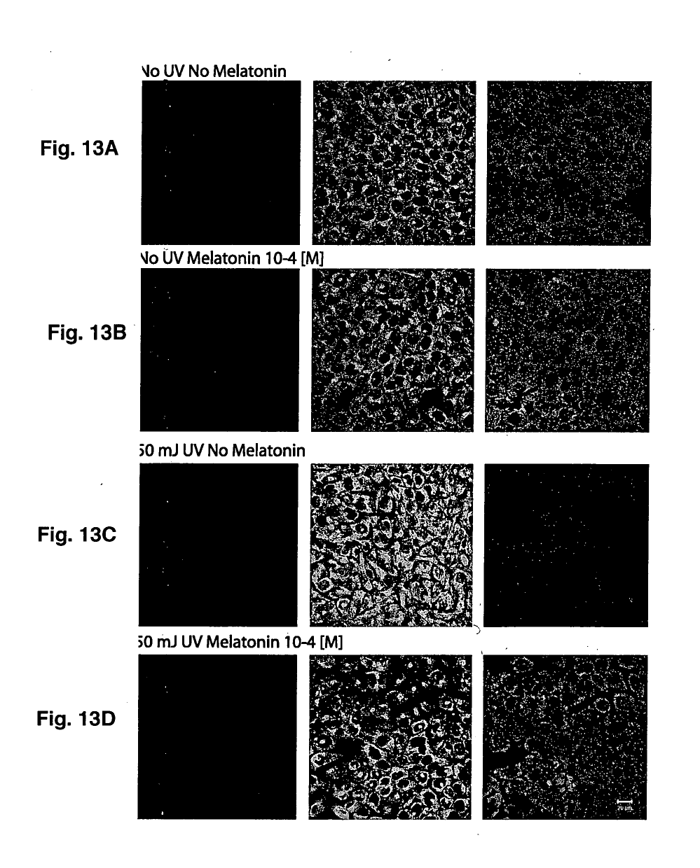

The JC-1 probe showed intense red fluorescence, co-localised with mitochondria in non-irradiated, non-melatonin treated cells representing physiological membrane potential (control) (FIG. 13A). To test, whether melatonin alone (without additional UV-exposure) would have influence on mitochondrial membrane potential, cells were incubated with melatonin at the concentration of 10−4 M and no change of mitochondrial membrane potential was observed (FIG. 13B). Irradiation with 50 mJ/cm² in normal non pre-treated keratinocytes showed attenuation of red fluorescence and elevated green fluorescence (mostly cytoplasmatic) indicative for loss of mitochondrial membrane potential (FIG. 13C). Preincubation with melatonin at the concentration of 10⁻⁴ M preserved mitochondrial membrane potential indicative for reduced UV-induced damage in the mitochondria (FIG. 13D). The changes in concentration of aggregated form of JC-1 (red, left panel) were in agreement with accumulation of JC-1 monomer (green, middle panel) and were also expressed as a ratio red/green shown in blue (FIG. 13, right panel).

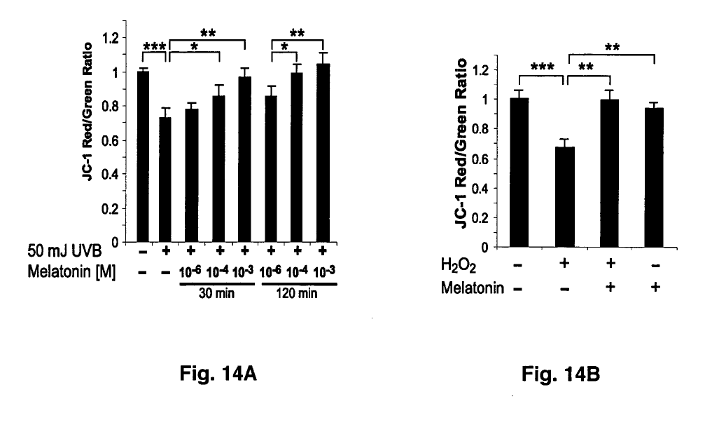

In order to support microscopic observation ratio between J monomer/J-aggregate, fluorescence ratio (red/green) was calculated and results were compared. UV irradiation lead to significant mitochondrial membrane potential reduction (ΔΨ) that was attenuated by melatonin in a dose dependent manner as shown by ratio between J monomer/J-aggregate fluorescence. Statistical evaluation of J monomer/J-aggregate ratios revealed significant differences for melatonin at the concentration of 10⁻⁴ and 10⁻³ M. Protective effects of melatonin were slightly stronger when incubation was performed for 120 min compared to 30 min (FIG. 14A). As a control for oxidative stress and reference to UV-induced oxidative mitochondrial damage the influence of H₂O₂ on mitochondrial potential (ΔΨ) was tested. H₂O₂ lead to a significant reduction of mitochondrial membrane potential, which was significantly counteracted by melatonin at the concentration of 10⁻⁴ M (FIG. 14B). Melatonin alone (without prior UV irradiation) did not influence mitochondrial membrane potential.

### EXAMPLE 18

### Melatonin Inhibits Activation of Caspases and PARP after UV Irradiation

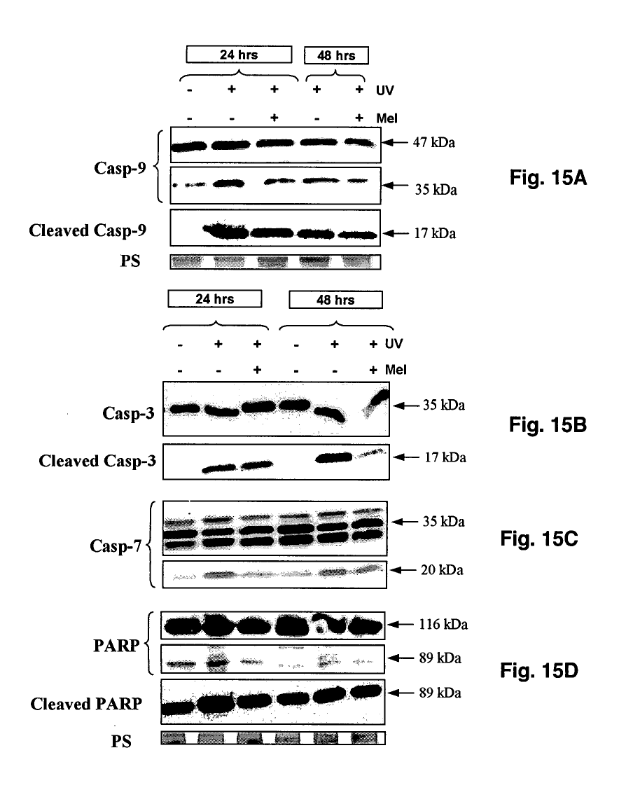

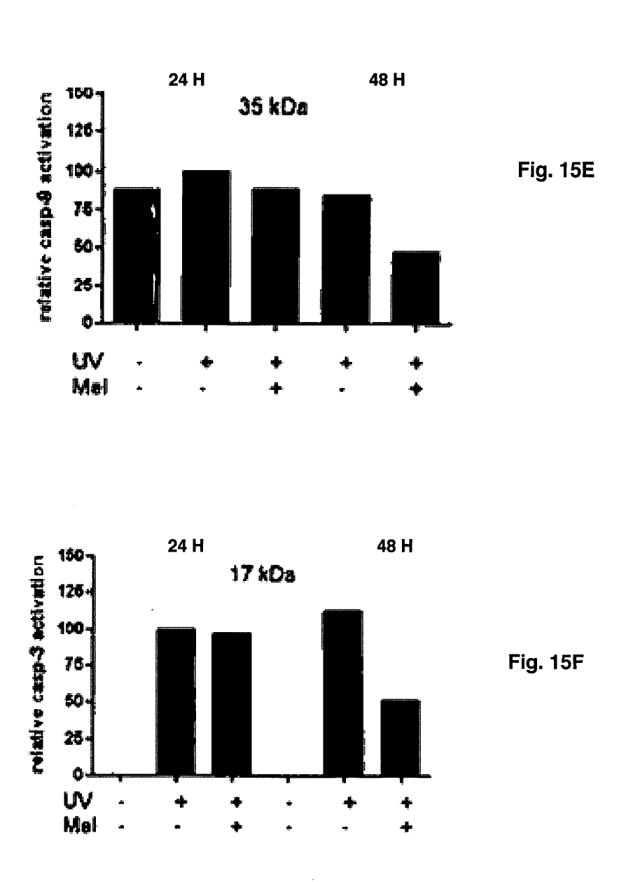

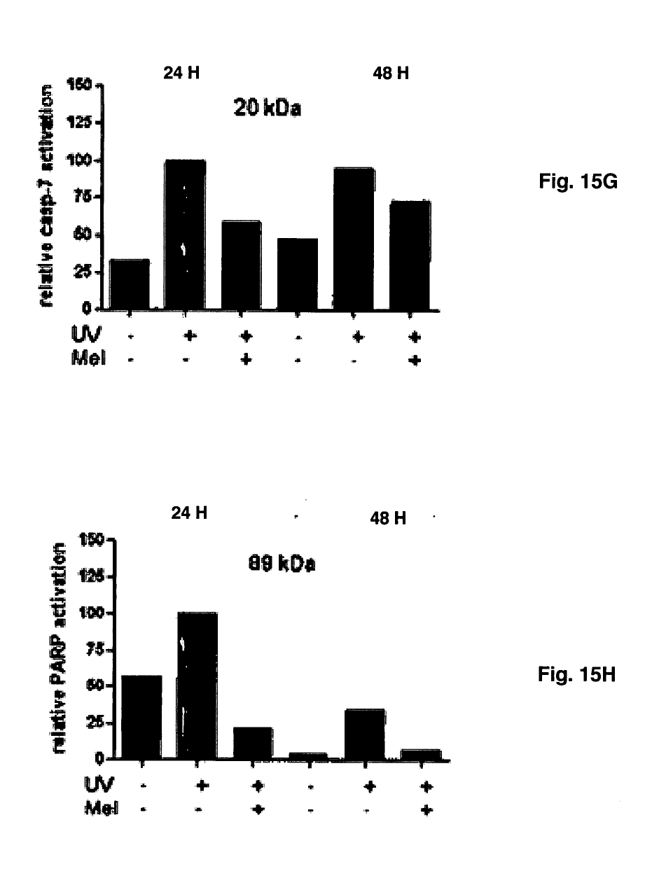

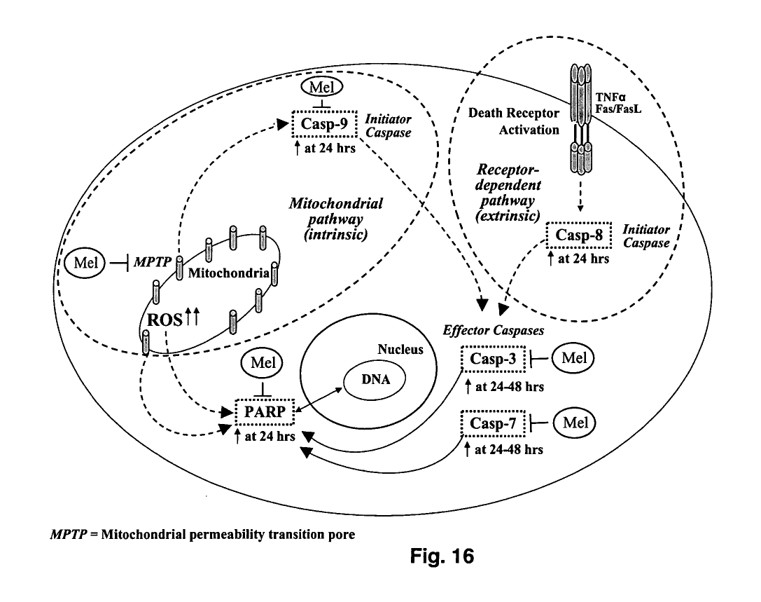

Activation of Casp-8 was observed 24 hrs after UV irradiation as shown by the 57 kD product and presence of its cleaved (activated) form of 43 kDa, however, melatonin treatment was without any effect on this process (data not shown). Casp-9 was strongly activated at 24 hrs after UV irradiation and melatonin treated samples showed weaker expression of its cleaved forms of 35 and 17 kDa (FIG. 15A, 15E). Notably, cleavage products of casp-9 were still present, even though at a lower level, after 48 hrs. The specific antibody against the effector caspase casp-3 detected a product of 35 kDa and the specific antibody for cleaved casp-3 detected the activated form of 17 kDa. 48 hours after UV-treatment further cleavage of Casp-3 was observed with clear reduction by melatonin (FIG. 15B, lower panel, 15F). The antibody against the effector caspase casp-7 detected the relevant protein at similar level of expression at 24 and 48 hrs after irradiation (FIG. 15C, 15G). The specific antibody against the cleaved form of casp-7 showed positive staining for the 20 kDa product which was reduced in melatonin treated keratinocytes when compared to non-melatonin treated samples. The antibody against poly-ADP-ribose-polymerase (PARP) detected the protein at 24 hrs after UV exposure and again its level was reduced in melatonin treated cells vs. untreated controls (FIG. 15D, 15H). Cleaved PARP was detected as a 89 kDa protein with PARP antibody and the specific antibody against cleaved form of PARP (FIG. 15D, lower panel) At both time points, the melatonin treated samples showed inhibition of expression and cleavage of the protein when compared to the control samples.

The following references are cited herein:
1. Arendt, J. (1988) Melatonin. Clin. Endocrinol.(Oxf) 29, 205-229
2. Lerner et al., (1958) J Am Chem Soc 80, 2587
3. Bubenik, G. A. (2002) Dig Dis Sci 47, 2336-2348
4. Cahill, et al., (1992) Vis Neurosci 8, 487-490
5. Tan, et al., (1999) Biochim. Biophys. Acta 1472, 206-214
6. Carrillo-Vico, et al., (2004) Faseb J 18, 537-539
7. Itoh, et al., (1999) Mol Hum Reprod 5, 402-408
8. Reiter, et al., (2002) Ann N Y Acad Sci 957, 341-344
9. Slominski, et al., (2005) Endocrine 27, 137-148
10. Slominski, A., Wortsman, J., and Tobin, D. J. (2005) FASEB J 19, 176-194
11. Tan et al., (1993) Endocr J 1, 57-60
12. Tan, et al., (2003) J Pineal Res 34, 75-78
13. Tan, et al., (2002) Curr. Top. Med. Chem. 2, 181-197
14. Maestroni, G. J. (2001) Expert. Opin. Investig. Drugs 10, 467-476
15. Reiter, et al., (1994) Ann. N. Y . Acad. Sci. 719, 1-12
16. Karasek, M., and Reiter, R. (2002) Neuroendocrinol Lett 23 Suppl 1, 14-16
17. Karbownik, M. (2002) Neuroendocrinol Lett. 23 Suppl 1, 39-44
18. Reiter, et al., (2005) J Pineal Res 39, 215-216
19. Slominski, et al., (2002) Serotoninergic and melatoninergic systems are fully expressed in human skin. FASEB J 16, 896-898
20. Slominski, et al., (2002) FEBS Lett 511, 102-106
21. Slominski, et al., (2003) Eur J Biochem 270, 3335-3344
22. Slominski, et al., (2004) Biochim Biophys Acta 1680, 67-70
23. Slominski, et al., (1996) J Biol Chem 271, 12281-12286
24. Schallreuter, et al., (1994) Science 263, 1444-1446
25. Hasse, et al., (2004) J Invest Dermatol 122, 307-313
26. Slominski, et al., (2003) J Cell Physiol 196, 144-153
27. Hipler, et al., (2003) Skin Pharmacol Appl Skin Physiol 16, 379-385
28. Slominski, A., and Pruski, D. (1993) Exp Cell Res 206, 189-194
29. Kadekaro, et al., (2004) J Pineal Res 36, 204-211
30. Berman, B. (2001) Int J Dermatol 40, 573-576
31. Collins, et al., (2004) Semin Cutan Med Surg 23, 80-83
32. de Vries, et al., (2004) Eur J Cancer Prev 13, 387-395
33. Fischer, et al., (2001) J Pineal Res 31, 39-45
34. Fischer, et al., (2004) J Pineal Res 37, 107-112
35. Bangha, et al., (1997) Dermatology 195, 248-252
36. Dreher, et al., (1998) Br J Dermatol 139, 332-339
37. Fischer, et al., (2006) J Pineal Res 40, 18-26
38. Fischer, et al., (2004) Skin Pharmacol Physiol 17, 190-194
39. Kim, et al., (1999) Arch. Pharm. Res. 22, 143-150
40. Mayo, et al., (2005) J Neuroimmunol 165, 139-149
41. Onuki, et al., (2005) J Pineal Res 38, 107-115
42. Silva, et al., (2004) J Neuroimmunol 156, 146-152
43. Ressmeyer, et al., (2003) Redox Rep 8, 205-213
44. Maharaj, et al., (2002) J Pineal Res 32, 257-261
45. Tan, et al., (2001) Faseb J 15, 2294-2296
46. Kelly, et al., (1984) Biochem Biophys Res Commun 121, 372-379
47. Silva, et al., (2004) J Pineal Res 37, 171-175
48. Semak, et al., (2005) Biochemistry 44, 9300-9307
49. Ma, et al., (2005) Drug Metab Dispos 33, 489-494
50. Lerner, A. B., and Nordlund, J. J. (1978) J Neural Transm Suppl, 339-347
51. Rozov, et al., (2003) J Pineal Res 35, 245-250
52. Hardeland, et al., (1993) Neurosci Biobehav Rev 17, 347-357
53. de Almeida, et al., (2003) J Pineal Res 35, 131-137
54. Almeida, et al., (2004) J Pineal Res 36, 64-71
55. Schallreuter, et al., (2001) J Photochem Photobiol B 64, 179-184
56. Tan, et al., (2000) Free Radic Biol Med 29, 1177-1185
57. Hardeland, et al., (2005) Melatonin. Int J Biochem Cell Biol, DOI:10.1016/j.biocel.2005.1008.1020
58. Tesoriere, et al., (2001) Free Radic Res 35, 633-642
59. Ximenes, et al., (2005) J Biol Chem
60. Horstman, et al., (2002) Bioorg Chem 30, 371-382
61. Dellegar, et al., (1999) Biochem Biophys Res Commun 257, 431-439
62. Wong, et al., (1999) Rapid Commun Mass Spectrom 13, 407-411
63. Vakkuri, et al., (1987) Endocrinology 120, 2453-2459
64. Stasica, P., Panetli, P., and Rosiak, J. M. (2000) J Pineal Res 29, 125-127
65. Lisby, S. et al., (2005) Exp Dermatol 14, 349-355
66. Schwarz, et al., (2002) Nat Cell Biol 4, 26-31
67. Krutmann, J. (2001) Eur J Dermatol 11, 170-171
68. Costa, et al., (1995) J Pineal Res 19, 123-126
69. Nickel, A., and Wohlrab, W. (2000) Arch Dermatol Res 292, 366-368
70. Tan, et al., (1999) Life Sci. 65, 2523-2529
71. Skinner, D. C., and Malpaux, B. (1999) Endocrinology 140, 4399-4405
72. Conti, et al., (2000) J Pineal Res 28, 193-202
73. Bubenik, et al., (1999) J Pineal Res 26, 56-63
74. Finocchiaro, et al., (1991) Biochem J 280 (Pt 3), 727-731
75. Iuvone, et al., (2005) Prog Retin Eye Res 24, 433-456
76. Reiter, R. J., and Tan, D. X. (2003) J Pineal Res 34, 79-80
77. Slominski, et al., (1994) Murine skin as a target for melatonin bioregulation. Exp Dermatol 3, 45-50
78. Kobayashi, et al., (2005) FASEB J 19, 1710-1712
79. Fischer, et al., (2002) Skin Pharmacol Appl Skin Physiol 15, 367-373.
80. Fischer, et al., (2004) J Pineal Res 37, 107-112.
81. Fischer, et al., (2006) J Pineal Res 40, 18-26
82. Slominski, et al., (2005) Biochim Biophys Acta 1755, 90-106
84. Stander, S., and Schwarz, T. (2005) Am J Dermatopathol 27, 116-121
85. Poppelmann, et al., (2005) J Biol Chem 280, 15635-15643
86. Leverkus, et al., (1997) Exp Cell Res 232, 255-262

What is claimed is:

1. A method of protecting the skin from ultra-violet radiation, comprising:
applying topically an effective dose of a formulation comprising melatonin, wherein said application protects the skin from the damaging effects of ultra-violet radiation.

2. The method of claim 1, wherein said protection is due to scavenging of UVB-induced reactive oxygen species by melatonin.

3. The method of claim 1, wherein said protection is due to generation of melatonin photoproducts.

4. The method of claim 3, wherein said melatonin photoproducts are 6-hydroxymelatonin, 2-hydroxymelatonin, 4-hydroxymelatonin and N¹-acetyl-N²-formyl-5-methoxy-kynuramine.

5. The method of claim 4, wherein said photoproducts generated due to metabolism of melatonin protects the skin against lipid peroxidation, protein oxidation and DNA damage.

6. The method of claim 1, wherein said damaging effects of ultra-violet radiation comprise photoaging and cancer.

7. A method of reducing photodamage to the skin of a subject comprising administering a composition comprising melatonin to the subject in an amount effective to inhibit UVB-induced apoptosis of skin cells exposed to ultra violet radiation.

8. The method of claim 7, wherein said inhibition of the UVB-induced apoptosis comprises attenuation of the mitochondrial membrane potential reduction, reduced activation of caspase 9, 3 and 7, and reduced PARP activation.

9. The method of claim 8, wherein said reduced PARP activation by melatonin is due to the attenuation of mitochondrial damage and/or by reduction of the reactive oxygen species.

10. The method of claim 7, wherein the composition is a topical composition.

11. The method of claim 10, wherein the topical composition is in a form selected from the group consisting of a cream, gel, salve, lotion or spray.

12. A method of retarding development of skin cancer in an individual at risk comprising:
applying topically an effective dose of a formulation comprising melatonin; wherein said application retards development of skin cancer in said individual at risk.

13. The method of claim 12, wherein said protection is due to generation of melatonin photoproducts.

14. The method of claim 13, wherein said melatonin photoproducts generated are 6-hydroxymelatonin, 2-hydroxymelatonin, 4-hydroxymelatonin and N¹-acetyl-N²-formyl-5-methoxy-kynuramine.

15. The method of claim 14, wherein said melatonin metabolites scavenge UVB induced reactive oxygen species and/or inhibit UVB-induced apoptosis of skin cells exposed to ultra violet radiation.

16. The method of claim 15, wherein said inhibition of the UVB-induced apoptosis comprises attenuation of the mitochondrial membrane potential reduction, reduced activation of caspase 9, 3 and 7, and reduced PARP activation.

17. The method of claim 12, wherein the composition is a topical composition.

18. The method of claim 17, wherein the topical composition is in a form selected from the group consisting of a cream, gel, salve, lotion or spray.

19. The method of claim 12, wherein said skin cancer is from the group consisting of basal cell carcinoma, squamous cell carcinoma or melanoma.
